(19) United States
(12) Patent Application Publication
(10) Pub. No.: US 2013/0018300 A1
(43) Pub. Date: Jan. 17, 2013

(54) WOUND CARE SYSTEM

(71) Applicants: Claudia Hannelore Igney, Erlangen (DE); Rainer Hilbig, Eindhoven (NL); Robert Pinter, Lubeck (DE)

(72) Inventors: Claudia Hannelore Igney, Erlangen (DE); Rainer Hilbig, Eindhoven (NL); Robert Pinter, Lubeck (DE)

(73) Assignee: KONINKLIJKE PHILIPS ELECTRONICS N.V., EINDHOVEN (NL)

(21) Appl. No.: 13/637,771

(22) PCT Filed: Mar. 22, 2011
(86) PCT No.: PCT/IB2011/051187

§ 371 Date: Sep. 27, 2012

(30) Foreign Application Priority Data
Mar. 31, 2010 (EP) 10158736.8

(51) Int. Cl. A61M 37/00; A61M 1/00

(52) U.S. Cl. 604/23

(57) ABSTRACT

The present invention relates to a wound care system for treating a wound (14) of a patient comprising: a bathing unit (16) being designed to limit a volume (18) surrounding the wound (14) and sealing said volume (18) against the outer atmosphere, the bathing unit (16) comprising at least one gas inlet (28) and one gas outlet (30) being in fluid communication with said volume (18), a source (20) for nitric oxide being connectable to said gas inlet (28), a vacuum unit (34) being connectable to said gas outlet (30) for creating a negative pressure inside said volume (18), and a sponge (36) being placeable inside said volume (18).

### FIELD OF THE INVENTION

The invention relates to the field of wound care systems. More specifically, the invention relates to the field of wound care systems based on topical nitric oxide administration.

### BACKGROUND OF THE INVENTION

It is widely known to use nitric oxide (NO) in a variety of applications. Next to technical applications such as an intermediate in the Ostwald process for the synthesis of nitric acid from ammonia, especially several therapeutic applications using nitric oxide are known.

One of the most famous therapeutic applications of nitric oxide is the administration for patients having the Persistent Pulmonary Hypertension (PPHN) in Neonates. However, many comparable or other therapeutic applications are known and discussed for the use of nitric oxide. As an example, nitric oxide is used by the endothelium of blood vessels to signal the surrounding smooth muscle to relax, thus resulting in widening the blood vessels and therefore increasing blood flow. This leads to nitric oxide being particularly applicable for blood pressure disease. Further exemplary applications for nitric oxide are directed towards improving lung function and treating or preventing bronchoconstriction, reversible pulmonary vasoconstriction, or for treating or preventing arterial restenosis resulting from excessive intimal hyperplasia in mammal. Apart from that, the administration of nitric oxide is particularly useful for treatment of infected tissue e.g. to kill bacteria. This application mostly involves topical delivery of a source of nitric oxide containing gas to a skin surface containing infected tissue. This and the vasodilatation effect is especially useful in wound healing systems, or wound care systems, respectively.

The administration of nitric oxide in wound care systems, however, raises several problems which have to be solved. First of all, although the administration of nitric oxide is very useful, high concentrations may be harmful and have thus to be avoided. Consequently, the concentration of nitric oxide has to be kept below a defined value.

Furthermore, nitric oxide tends to oxidize to nitrogen oxides in higher oxidation states (NOₓ) in the presence of oxygen. For example, nitrogen dioxide (NO₂) may be formed. Due to the high toxicity of these nitrogen oxides in higher oxidation states, especially nitrogen dioxide, the formation of these compounds has to be avoided. Apart from that, if nitric oxide oxidizes, the concentration of the helpful nitric oxide decreases resulting in a deteriorated efficiency and helpfulness of the wound healing process.

Additionally, there are further demands with respect to wound healing processes which are not solved by nitric oxide therapy.

Known from US 2002/0156416 A1 is a device and method for treatment of surface infections with nitric oxide. The device comprises a source of nitric oxide gas, such as a gas cylinder, or a feed line of a wall supply, a bathing unit, a flow control valve, and a vacuum unit. The bathing unit is in fluid communication with the source of nitric oxide gas and is adapted for surrounding the area of infected skin. However, the problems discussed above are not completely solved. As an example, the oxidation of nitric oxide is not completely avoided. Especially the tubings used often allow an inflow of oxygen thereby causing nitric oxide to oxidize.

### SUMMARY OF THE INVENTION

It is an object of the invention to provide a wound care system, which enables to overcome at least one of the limitations as set forth above.

It is a further object of the invention to provide a wound care system which has an improved effectiveness.

It is a further object of the invention to provide a wound care system which limits oxidation of nitric oxide.

These objects are achieved by a wound care system for treating a wound of a patient comprising: a bathing unit being designed to limit a volume surrounding the wound and sealing said volume against the outer atmosphere, the bathing unit comprising at least one gas inlet and one gas outlet being in fluid communication with said volume, a source for nitric oxide being connectable to said gas inlet, a vacuum unit being connectable to said gas outlet for creating a negative pressure inside said volume, and a sponge being placeable inside said volume.

According to the invention, it is possible to combine two methods each having a beneficial effect with respect to wound healing. In detail, a system according to the invention combines the administration of nitric oxide with vacuum therapy.

Vacuum therapy (also called vacuum assisted closure, vacuum sealing or topical negative pressure therapy) is a very expedient process used for improving wound healing. It mainly comprises the use of vacuum assisted drainage to remove blood or serous fluid from a wound or operation site. Debridement and wound healing in many different types of wounds may be accelerated by the application of controlled levels of negative pressure. It is believed that the negative pressure assists with removal of interstitial fluid, decreasing localized oedema and increasing blood flow, which, in turn, decreases tissue bacterial levels. Therefore, infections are avoided. The underpresure additionally stimulates cell growth and has thus a beneficial influence to the wound healing process. Apart from that, the generation of a negative pressure in the volume in combination with a sponge ensures that the entire surface area of the wound is uniformly exposed to this negative pressure effect, prevents occlusion of the perforations in the drain by contact with the base or edges of the wound, and eliminates the theoretical possibility of localized areas of high pressure and resultant tissue necrosis. In fact, vacuum therapy may shorten the wound healing period or even allows curing types of wounds, an improved healing process of which is not known with respect to a wound healing without vacuum therapy. However, these are just exemplary advantages of the vacuum therapy. More beneficial effects may be reached.

Next to said beneficial influence with respect to wound healing, the provision of a vacuum leads to the advantage that the tendency of nitric oxide to oxidize is strongly reduced in an atmosphere of reduced pressure. Thus, neither effective nitric oxide is lost, neither harmful nitrogen oxides are formed in the vicinity of the wound.

The opportunity of providing both nitric oxide therapy as well as vacuum therapy combines the beneficial effects of the two therapies in one single system. The system is thus much more efficient by the fact that it may be chosen between vacuum therapy or nitric oxide administration or a combination of the latter to be used. It is thus possible to benefit from the advantages of nitric oxide administration as well as vacuum therapy leading to a broader range of wound healing improvement.

In a second aspect of the invention, a wound care system for treating a wound of a patient is provided comprising: a bathing unit being designed to limit a volume surrounding the wound and sealing said volume against the outer atmosphere, the bathing unit comprising at least one gas inlet and one gas outlet being in fluid communication with said volume, a source for a gas comprising nitrogen, and a source for a gas comprising oxygen, or a source for a gas comprising a mixture of nitrogen and oxygen for creating a mixture of nitrogen and oxygen in said volume, and a heating element being placeable inside that volume.

With regard to this embodiment, it is possible to generate the nitric oxide in direct vicinity to the wound to be treated. Due to the provision of a gas mixture in the volume comprising both nitrogen and oxygen, an atmosphere is created which is well applicable for the generation of nitric oxide. The heating element may thus heat up the gas mixture in the volume causing the nitrogen to react with oxygen to form nitric oxide. This enables the generated nitric oxide to directly influence the healing process in a helpful way. The risk of nitric oxide to be oxidized before being conveyed to the wound is thus minimized, or completely avoided.

Furthermore, the concentration of the nitric oxide may be adjusted by the ratio of nitrogen and oxygen being present in the volume. With this regard, one possible way to adjust the required gas ratio is by providing one single gas source dispensing a gas mixture comprising oxygen and nitrogen in the desired amount.

In a preferred embodiment of the invention, the bathing unit is formed as a flexible foil. This enables a very fast and easy handling of the bathing unit when fixing it around the wound of the patient and when removing the foil. Such a bathing unit may thus be handled independent of the size of the wound and the place, where the wound appears. Furthermore, due to the good handability, the bathing unit may be used by persons not being teached in the medical sector and is thus very well usable in home care applications. These advantages may especially be achieved if the foil is formed of an organic polymer. These foils are furthermore easy to form in a cost-saving manner and stable enough for the desired application. A fracture or other damages of the bathing unit may thus be avoided.

In a further preferred embodiment of the present invention, the bathing unit is covered with an antibacterial coating. This avoids bacteria accumulating especially to the inner side of the bathing unit and thus counteracting the healing process.

In a further preferred embodiment of the present invention, the bathing unit is covered with a non-adherent coating. This prevents the bathing unit from sticking to the sponge, or to the skin of the patient. The bathing unit is thus easily to be removed after a therapy step. An injury due to a sticky bathing unit may therefore be avoided.

In a further preferred embodiment, a membrane is provided being designed to decrease the amount of oxygen being present inside the volume. Oxidation of the therapeutic useful nitric oxide to nitrogen oxides in higher oxidation states is thus reduced or completely avoided at least in the vicinity of the wound to be treated. Therefore, no useful nitric oxide is lost whereas the formation of harmful nitrogen oxide is avoided or at least, the concentration of harmful nitrogen oxides is hold under a harmful level. Furthermore, a membrane is useful due to the fact that air may be used as gas mixture comprising nitrogen and oxygen. In this case, the membrane may decrease the oxygen content to an amount in which an appropriate reaction of nitrogen and oxygen may occur.

In a further preferred embodiment of the present invention, the membrane is located at the gas inlet of the bathing unit. With this regard, the membrane is located directly upstream the volume, leading to the oxygen being reduced directly before the gas enters said volume. It may thus securely be avoided, that oxygen enters the volume, even if tubings are used for connecting the nitric oxide gas source with the volume. Furthermore, the amount of oxygen may be reduced to an amount at which the reaction of nitrogen and oxygen is well applicable and the concentration is hold in a constant range. Furthermore, especially, if the membrane is designed as a plug in the gas inlet, due to its size being limited to an extend which is sufficient to achieve the desired effect but not larger than needed, the bathing unit may be formed in a very cost-saving manner.

In a further preferred embodiment of the present invention, the membrane 44 forms part of the bathing unit 16. This is a very cost-saving way of forming the system according to the invention being equipped with a membrane, because this arrangement enables the membrane 44 to form the gas inlet. Thus, no further gas inlet has to be provided.

In a further preferred embodiment of the present invention, the membrane has a sorptive affinity for oxygen. This securely allows to remove oxygen from the gas and thus to enable an atmosphere in the volume having a reduced oxygen content. This arrangement is especially useful if the membrane is located inside the volume.

In a further preferred embodiment of the present invention, a dispose unit is arranged downstream the gas outlet. This arrangement allows eliminating the nitric oxide gas. With this regard, an oxidation to harmful nitrogen oxides in higher oxidation states outside the bathing unit is prevented making it much or secure to handle.

### BRIEF DESCRIPTION OF THE DRAWINGS

These and other aspects of the invention will be apparent from and elucidated with reference to the embodiments described hereinafter.

In the drawing:

FIG. 1 shows a schematical view of a system according to the first aspect of the present invention.

FIG. 2 shows a schematical view of a system according to the second aspect of the present invention.

FIG. 3 shows a schematical view of a further embodiment of a system according to the second aspect of the present invention.

FIG. 4 shows a schematical view of a system according to a combination of the first and second aspect of the invention.

### DETAILED DESCRIPTION OF EMBODIMENTS

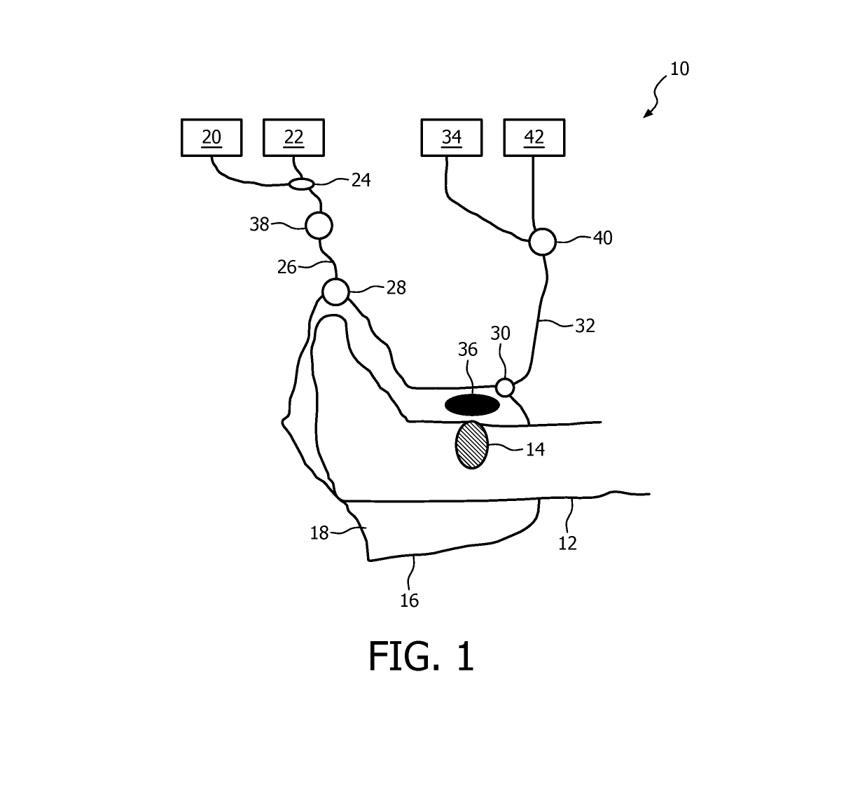

A wound care system 10 according to the first aspect of the present invention is shown in FIG. 1. The system 10 according to the invention is particularly suitable for the administration of nitric oxide in combination with vacuum therapy for wound healing processes.

In FIG. 1, a part 12 of the skin of a patient is schematically shown. At that part 12, the patient has a wound 14 which may be every kind of wound which should be treated for an improved wound healing procedure. In particular, the wound 14 may be an open wound or skin with infected tissue. In order to improve the wound healing process, administration of nitric oxide to the wound 14 is advantageous. As an example, the recovery time of both aseptic and purulent wounds may be shortened due to the nitric oxide administration. This effect possibly may be explained by the fact that wound healing processes itself are known to involve a sharp increase of nitric oxide generation in wound tissue caused by the activation of constitutive isoforms of NO-synthase and by a markedly enhanced synthesis of inducible NOS (iNOS).

For administering nitric oxide, a bathing unit 16 is provided. The bathing unit 16 limits a volume 18 being in direct vicinity to the wound 14 and surrounding the latter. Inside the volume 18, nitric oxide may be administered to the wound 14 to achieve the desired improved healing effect. Therefore, the bathing unit 16 seals the volume 18 against the outer atmosphere in an air tight manner. This avoids an air exchange between the gas located inside the volume 18 and the atmosphere being outside the volume 18.

The bathing unit 16 may be formed of any suitable material. It is thus possible to use a rigid bathing unit 16 having a defined shape. However, it is most preferred that the bathing unit 16 is formed as a flexible foil. This enables a very easy way of handling, i.e. of applying the bathing unit 16 to the patient before and removing it after a therapy step. The bathing unit 16 just has to be wrapped around the skin of the patient such, that it surrounds the wound 14. With this regard, it is especially advantageous that the bathing unit 16 is formed of an organic polymer which may lead to a very flexible foil. An exemplary polymer to form the foil is polyurethane. With this regard, a special sealing is not required, as a flexible foil as such enables an air tight sealing with a patients skin, when being firmly secured to the healthy skin around the wound margin. However, an additional sealing may be appropriate by using e.g. an adhesive tube, or adhesive foil, respectively.

The bathing unit 16 may furthermore be equipped with an antibacterial and/or with a non-adhesive coating at least at its inner side. This avoids bacteria being potentially present in the gas fed into the volume 18 to contaminate the bathing unit 16 and therefore counteracting the healing process. Additionally, the non-adhesive coating prevents the bathing unit 16, or the foil, respectively, from sticking to the skin or to the sponge. A removal of the foils may thus be improved, and injuries due to a sticky bathing unit 16 may be avoided.

For conveying nitric oxide into the volume 18, the latter is in fluid communication with a nitric oxide gas source 20. The nitric oxide gas source 20 may be any kind of gas source which is appropriate. As an example, a pressurized gas cylinder containing NO-gas may be used. The system will then be usable for home care applications. However, a feed line being connected to a wall supply may as well be used. This arrangement is especially suitable for applications in a hospital. The nitric oxide gas source 20 may be connected to a pump, or compressor unit, respectively, which may convey the nitric oxide gas from its source to the volume 18. However, this compressor unit may be omitted if a pressurized gas cylinder is used as nitric oxide gas source 20. Furthermore, an air-plasma-NO-generator may be appropriate.

Preferably, a pressure regulator is provided to regulate the pressure to a constant flow. The pressure regulator may ensure to have a defined gas pressure without fluctuations, which is especially desirable in medical applications.

Optionally, a flow control valve, like a mass flow controller, is provided to exactly adjust the amount of nitric oxide flowing to the patients wound 14. It is then positioned upstream the bathing unit 16 but downstream the nitric oxide gas source 20.

Due to the fact, that nitric oxide is especially advantageous in small concentrations, it is mostly preferred that a gas source 20 is provided which comprises nitric oxide in concentrations of ≧50 ppm to ≦1200 ppm, wherein it is especially advantageous that the nitric oxide concentration is present in the nitric oxide gas source 20 in an amount of ≧100 ppm to ≦800 ppm, in particular ≧150 ppm to ≦500 ppm, together with an inert gas.

However, it is as well possible to use a gas source 20 delivering pure nitric oxide. In this case, it is possible to dilute the nitric oxide with an inert gas, e.g. nitrogen, before conveying it into the volume 18. Therefore, an additional inert gas source 22 is required, being in fluid communication to the stream of pure nitric oxide gas upstream the bathing unit 16. Thus, the nitric oxide may be diluted before administration to the patient's skin. With this regard, it is preferred to provide a gas blender 24 for creating a mixture of the nitric oxide gas together with the diluting gas upstream the bathing unit 16.

In order to create a fluid communication between the volume 18 and the nitric oxide gas source 20, a tubing 26 may be provided, which is connected to gas source, or the blender 24, respectively at its one side, but which is furthermore connected to a gas inlet 28 of the bathing unit 16. Substantially, the gas inlet 28 is an aperture which is permeable for gas, thereby allowing to convey gas into the volume 18. The gas inlet 28 may comprise a one way valve to prevent backflow of the gas into the tubing 26.

Apart from the gas inlet 28, the bathing unit 16 furthermore comprises a gas outlet 30, through which the nitric oxide gas is guided to the outside of the volume 18. The gas inlet as well as the gas outlet 30 are preferably locates such, that the nitric oxide flowing through the volume 18 has a long residence time. This improves the efficiency of the nitric oxide administration.

The gas outlet 30 may be connected to a flexible tubing 32, the latter being in flow connection to a vacuum unit 34. The vacuum unit 34 is designed to create a negative pressure inside the volume 18. On the one hand, the negative pressure further reduces the amount of nitric oxide being oxidized to nitrogen oxides in higher oxidation states. Furthermore, the vacuum may be used to create an underpresure inside the volume 18 causing the nitric oxide gas to be sucked into the volume 18.

According to the invention, furthermore a sponge 36 is provided inside the volume 18. The term “sponge”, as used herein, shall refer to a foam-like piece of material having a porous structure, or open-cell structure, respectively. The sponge 36 is designed to cover the whole wound. Due to its porosity, the sponge 36 may receive blood or serous fluid from the wound 14. The sponge 36 may exemplarily be formed of polyurethane or polyvinyl alcohol, depending on the wound to be treated. In case a polyurethane sponge is used the latter is hydrophobic and may be large pored. In case a polyvinyl alcohol sponge is used, the latter is hydrophilic and may have small pores. When exposed to a vacuum, fluid is drawn from the wound 14 through the foam into an optional reservoir for subsequent disposal. However, the reservoir is not strictly necessary, as most of the fluid may be stored in the open-cell structure of the sponge 36. Thus, it depends from the state of wound healing and therefore from the amount of fluid formed in the wound, if a reservoir is needed. Consequently, in an advanced state of wound healing, the reservoir may be omitted.

In combination with the vacuum unit 34, the sponge 36 allows applying vacuum therapy by using the system according to the invention. Thus, a combination of a therapy based on nitric oxide administration and on vacuum therapy is enabled.

In order to allow a wide variety of possible healing methods, valves may be provided between the source of nitric oxide 20, or the blender 24, respectively, and the gas inlet 28, namely the so called inlet valve 38, as well as between the gas outlet 30 and the vacuum unit 34, namely the so called outlet valve 40. Consequently, the valves 38, 40 may be located in the tubing 26 and in the tubing 32, respectively. The system according to the invention thus allows to operate in different therapy modes.

Firstly, it is possible to only use nitric oxide therapy. In this case, the inlet-valve 38 is in an open position with respect to the nitric oxide gas source 20, or the blender 24, respectively. This position of the inlet valve 38 allows the nitric oxide gas to enter the volume 18. The flow of the nitric oxide gas may then exemplarily be affected by a pressurized gas cylinder or by a compressor unit. The outlet valve 40 may in this case be in a closed position with respect to the vacuum unit 34. This allows the gas stream which leaves the bathing unit 16 to be conveyed into a dispose unit 42. The dispose unit 42 is a device in which nitric oxide is eliminated. In detail, nitric oxide may be hydrolyzed to nitric acid or it may be adsorbed by an adsorbent. However, it is advantageous that no nitric oxide leaves the system as the oxygen which is present in the atmosphere would oxidize the nitric oxide to form harmful nitrogen oxides in higher oxidation states.

Nitric oxide therapy as such may furthermore be applied by using the inlet valve 38 in an open position with respect to the nitric oxide gas source 20, or the blender 24, respectively, and the outlet valve 40 in an open position with respect to the vacuum unit 34. In this regard, the vacuum unit 34 can be adjusted to withdraw nitric oxide containing gas at substantially equal rate as the gas is delivered to the bathing unit 16.

Additionally to applying nitric oxide therapy as such, the system according to the invention furthermore enables to apply vacuum therapy to the wound 14. Therefore, the inlet valve 38 is closed with respect to the nitric oxide gas source 20, or the blender 24, respectively, whereas the outlet valve 40 is opened with respect to the vacuum unit 34. This allows to reduce the pressure in the volume 18 and to create an underpresure inside the latter. Appropriately, the vacuum ranges at 75 to 400 mmHg, in particular at 75-150 mm Hg, depending on the type of wound 14 to be treated. With this regard, the bathing unit 16 is inflated leading to the sponge 36 being pressed against the wound. Consequently, the vacuum unit 34 together with the sponge 36 allows the application of wound therapy. In this regard, a dispose unit 42 may be provided downstream the vacuum unit 34 to eliminate the nitric oxide gas. Dependent from the therapy mode, the position of the dispose unit 42 may be chosen. However, if a dispose unit 42 is provided, it has to be located downstream the gas outlet 30.

The vacuum therapy may be applied in a continuous manner, or in an intermittent manner. In a continuous manner, the vacuum may be formed like described above. However, in an intermittent manner, periods of vacuum therapy like described above are alternated with periods with atmospheric pressure. Therefore, a further gas inlet may be provided through which air may flow into the volume 18. This gas inlet may be located directly at the bathing unit 16, or at the tubing 26.

However, especially when applying the vacuum therapy in an intermittent manner, the system according to the invention enables to apply an alternating vacuum therapy and nitric oxide administration. This may be realized by venting the volume 18, after a period of vacuum therapy, with nitric oxide gas. The valves 38, 40 have to be adjusted in a corresponding manner. This allows to work with a therapy that combines both the advantages of nitric oxide administration as well as the benefits of the vacuum therapy.

Advantageously, a control unit may be provided which controls the administration of nitric oxide and the creation of a vacuum in defined time ranges. This enables to work with either single therapies or by means of a combined therapy e.g. over night. Furthermore, the control unit may be adjusted from medical staff, like a nurse or a medical doctor. This secures the treatment with an adequate therapy in an adequate duration even when the system is used in home care applications where medical staff is not present.

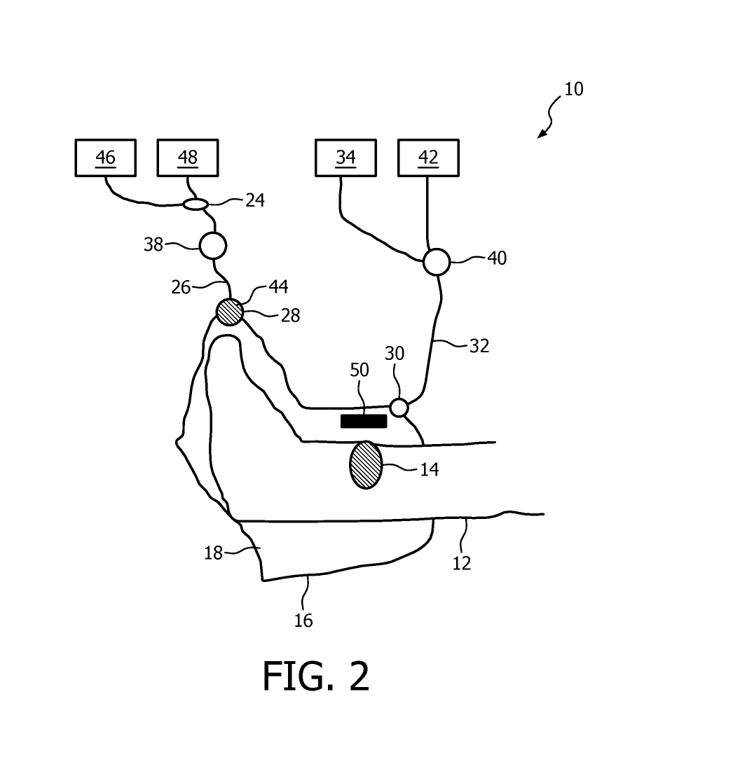

An embodiment with respect to a wound care system according to the second aspect of the invention is shown in FIG. 2.

Similar to FIG. 1, a part 12 of the skin of a patient is schematically shown, the part 12 comprising a wound 14. In order to improve the wound healing process, administration of nitric oxide to the wound 14 should be applied. For administering nitric oxide, the bathing unit 16 is provided. The bathing unit 16 limits the volume 18 being in direct vicinity to the wound 14 and surrounding the latter. Inside the volume 18, nitric oxide may be administered to the wound 14 to achieve the desired improved healing effect. Therefore, the bathing unit 16 seals the volume 18 against the outer atmosphere in an air tight manner. This avoids an air exchange between the gas located inside the volume 18 and the atmosphere being outside the volume 18.

Again, it is most preferred that the bathing unit 16 is formed as a flexible foil, in particular formed of an organic polymer like polyurethane, leading to the advantages discussed with respect to FIG. 1. Consequently, it is advantageous to equip the foil with an antibacterial and/or with a non-adhesive coating at least at its inner side.

According to the second aspect of the invention, the system is designed to form nitric oxide in direct vicinity to the wound 14 and thus inside the volume 18. Therefore, a gas mixture has to be present in the volume 18 which comprises nitrogen and oxygen. For providing said gas mixture inside the volume 18, the latter may be in fluid communication with a source 46 of a gas comprising nitrogen and with a source 48 of a gas comprising oxygen. These gas sources 46, 48 may be pressurized gas cylinders or wall supplies. To generate a mixture of nitrogen and oxygen, the gas sources 46, 48 may be in fluid communication with a gas blender 24. Downstream the gas blender 24, the gas mixture is conveyed through a tubing 26 to the gas inlet 28, to enter the volume 18, e.g. by means of a compressor, or by means of the pressurized gas cylinders.

Alternatively, one single gas source may be provided which dispenses a mixture of nitrogen and oxygen. As an example, this may be a pressurized gas cylinder comprising said gas mixture. In this case, the gas cylinder may be in fluid communication with the gas inlet 28, e.g. via tubing 26. Again, the gas may be conveyed either via the pressurized gas cylinder, or by means of a compressor unit. Furthermore, it might be possible to use a mixture of nitrogen and air. With this regard, a pressurized nitrogen gas cylinder may be used being in fluid communication with the gas inlet 28, e.g. via a tubing. Moreover, an additional gas inlet may be provided which is in fluid communication with the volume 18, or with the tubing connecting the nitrogen gas cylinder with the gas inlet 28. In this case, a gas mixture may be provided having a decreased oxygen concentration, which provides an improved atmosphere for forming nitric oxide.

It is, however, especially advantageous that the mixture of nitrogen and oxygen comprises ≦5% of oxygen. With this regard, most, or all of the oxygen is used for oxidation of nitrogen. Thus, substantially no oxygen is left for oxidizing nitric oxide. With this regard, the generation of harmful nitrogen oxides in higher oxidation states may be avoided.

Again, a pressure regulator, and/or a flow control valve may be advantageous to secure that an appropriate flow of gas is conveyed to the volume 18, and a one way valve may prevent backflow of the gas being present in the volume 18 to the outside of said volume 18.

Additionally, according to the second aspect of the invention, a heating element 50 is provided inside the volume 18. The heating element 50 may be designed as a flat metallic sheet or pin. It may be heatable by electric current. In this case, electric connectors are provided to connect the heating element 50 with a source of electric current, e.g. a battery. The heating element 50 is designed to heat the gas located inside the volume 18. This enables to increase the temperature of the gas to a value at which nitrogen may react with oxygen to form nitric oxide.

Due to the provision of the heating element 50 it is thus possible to form the desired nitric oxide in direct vicinity to the wound 14. This results in an increased concentration of nitric oxide at the wound 14 leading to an effective utilization of the provided nitric oxide gas.

With respect to nitric oxide therapy, nitric oxide gas tends to oxidize to nitrogen oxides in higher oxidation states. In order to reduce, or to avoid such an oxidation, the system according to the invention may, in an especially preferred embodiment of the present invention, comprise a membrane 44. The membrane 44 is designed to decrease the oxygen content inside the volume 18. The membrane 44 may be formed from a polymeric fiber. However, there are several possibilities to form such a membrane 44 known in the art. The decrease of the oxygen content reduces the risk of nitric oxide being oxidized. This has the advantage that no nitric oxide is lost and thus all nitric oxide which is present inside the volume 18 may be used to improve the healing process. Due to the provision of the membrane 44, the improvement of the healing process may thus be much more effective. Furthermore, toxic gases like nitrogen dioxide will not occur or at least they will stay under harmful levels.

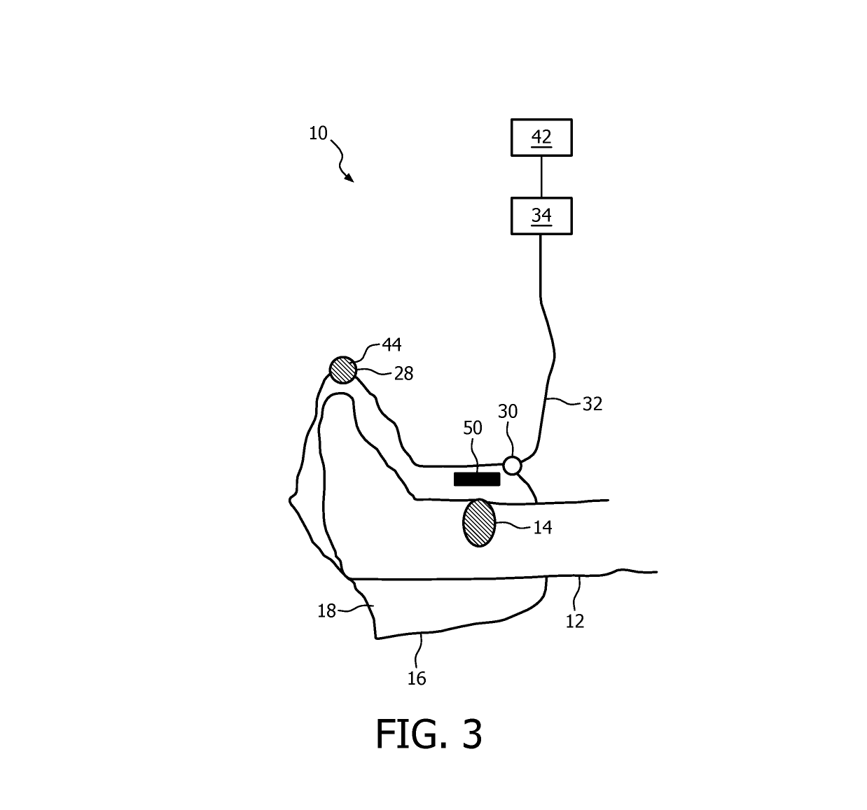

The effect of decreasing the oxygen content may be realized by the membrane 44 removing the oxygen from the gas flow entering the volume 18 through the gas inlet 28. A simple way to form this membrane 44 is to use a membrane 44 that has decreased permeation properties with respect to oxygen but well permeates other gases, especially nitrogen. With this regard, the membrane 44 may be designed as a plug in the gas inlet 28 or in the tubing 26. It is furthermore possible to design the membrane 44 as a flat sheet, or as part of the bathing unit 16, located at the gas inlet 28, like described with respect to FIG. 3. Consequently, the membrane may have a size which corresponds to the size of the gas inlet 28.

Instead of using permeation properties of the membrane 44 with respect to reducing the oxygen content in the volume 18, the membrane 44 may also be designed to decrease the oxygen content by adsorption of oxygen. This leads to oxygen being present in the volume 18 to be adsorbed by the membrane 44 and thus to be prevented from oxidizing the nitric oxide gas.

This ensures that the gas flows through the membrane 44 before entering the volume 18 and allows reducing the oxygen content of the gas mixture to an amount in which an appropriate reaction of nitrogen and oxygen may occur.

It may preferably adsorb the oxygen, thereby reducing the concentration of the latter. Again, to circumvent oxidation of nitric oxide, it is especially advantageous, if the membrane 44 is designed to reduce the amount of oxygen to ≦5%.

Downstream the volume 18, a gas outlet 30 is provided for an outflow of the gas mixture, and/or the generated nitric oxide. The gas outlet 30 is in fluid communication with a vacuum unit 34 and/or a dispensing unit 42. This allows to eliminate the formed nitric oxide gas and furthermore to withdraw the gas out of the volume 18.

Comparable to the first aspect of the invention, an inlet valve 38 as well as an outlet valve 40 may be provided to allow a wide variety of possible healing methods. As an example, inlet valve 38 may be opened with respect to the respective gas sources, whereas outlet valve 40 is closed with respect to the vacuum unit 34 but open with respect to the dispose unit 42. In this case, the gas mixture may be conveyed by means of a compressor unit or by means of the pressurized gas cylinders, and may flow into the bathing unit 16. Due to the heating element 50 heating the gas inside the volume 18, nitrogen will be oxidized to form nitric oxide, thereby improving the healing effect. Downstream the volume 18, the gas will leave the bathing unit 16 through the gas outlet 30 and will be conveyed through the dispose unit 42 to the outer atmosphere.

In another mode of operation, the inlet valve 38 may be in the position like stated above, but the outlet valve 40 is in an open position with respect to the vacuum unit 34. In this case, the gas may be conveyed by means of the vacuum unit 34. In order to eliminate the nitric oxide gas, it is advantageous to provide a dispose unit downstream the vacuum unit 34.

Especially with respect to home care applications, however, the usage of the vacuum unit 34 is especially preferred, in case the single source for the gas mixture is the atmosphere surrounding the bathing unit 16, i.e. air. In this case, it is taken advantage of the fact that air itself mainly comprises nitrogen and oxygen, wherein the major amount is nitrogen. With this regard, the vacuum unit 34 may suck the air into the volume 18 through the gas inlet 28.

This embodiment is shown in FIG. 3. According to FIG. 3, the membrane 44 is designed as a flat sheet, or as a part of the bathing unit 16, or the foil, respectively. The gas inlet 28 may thus be designed as the region, where the membrane 44 is located. In other words, the membrane 44 may form the gas inlet 28. Therefore, by creating a negative pressure inside the volume 18, air is sucked into said volume 18, thereby creating a mixture of nitrogen and oxygen inside the volume 18. Due to the provision of the membrane 44, the atmosphere inside the volume 18 mainly comprises nitrogen. Oxygen is as well present, but, however, in a minor amount. In case, the heating element 50 heats the atmosphere inside the volume 18 in the vicinity to the wound 14, nitrogen is oxidized to form nitric oxide. Due to the low concentration of oxygen in the gas mixture, nitric oxide is formed without itself being oxidized. Harmful nitrogen oxides in higher oxidation states are thus avoided. Downstream the volume 18, the gas mixture may leave the volume 18 through gas outlet 30, tubing 32 and through the vacuum unit 34 and the dispose unit 42.

This embodiment is especially advantageous for home care applications. No gas cylinder, wall supply, or the like are required. The system may be portable and provide a good handability.

It is apparent that the system according to the second aspect of the invention again allows several ways of therapy methods.

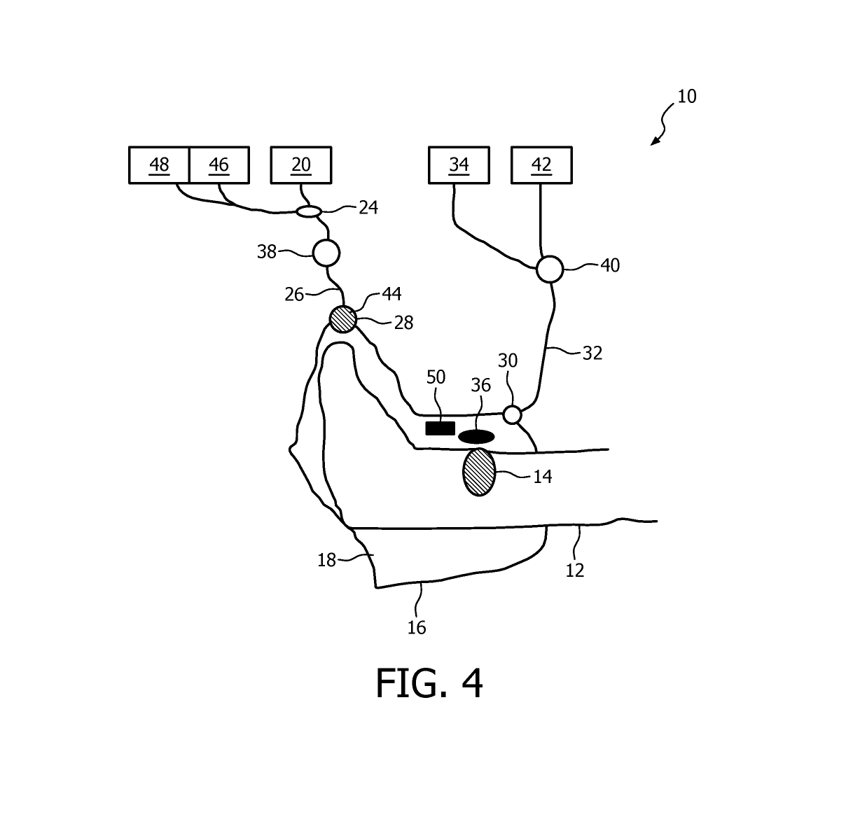

In a further embodiment of the invention, a combination of the first and second aspect of the invention is provided. With this regard, a preferred embodiment is shown in FIG. 4. According to FIG. 4, the system 10 according to the invention comprises both a source 20 for nitric oxide as well as a source 46 for nitrogen and a source 48 for oxygen, or a source for both nitrogen and oxygen, e.g. air in combination with a membrane 44 like described in FIG. 3. Additionally, both a sponge 36 and a heating element 50 are provided. Due to the periphery, or features like described with respect to FIGS. 1, 2 and 3, which may additionally be present, the system is usable in an even broader range of healing methods. It is thus possible to use the advantages of the first aspect of the invention in combination with the second aspect of the invention.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A wound care system for treating a wound (14) of a patient comprising:
a bathing unit (16) being designed to limit a volume (18) surrounding the wound (14) and sealing said volume (18) against the outer atmosphere, the bathing unit (16) comprising at least one gas inlet (28) and one gas outlet (30) being in fluid communication with said volume (18),
a source (20) for nitric oxide being connectable to said gas inlet (28),
a vacuum unit (34) being connectable to said gas outlet (30) for creating a negative pressure inside said volume (18), and
a sponge (36) being placeable inside said volume (18).

2. A wound care system for treating a wound (14) of a patient comprising:
a bathing unit (16) being designed to limit a volume (18) surrounding the wound (14) and sealing said volume (18) against the outer atmosphere, the bathing unit (16) comprising at least one gas inlet (28) and one gas outlet (30) being in fluid communication with said volume (18),
a source for a gas comprising nitrogen, and a source for a gas comprising oxygen, or a source for a gas comprising a mixture of nitrogen and oxygen for creating a mixture of nitrogen and oxygen in said volume (18), and
a heating element being placeable inside that volume (18).

3. System according to claim 1, wherein the bathing (16) unit is formed as a flexible foil.

4. System according to claim 1, wherein the bathing unit (16) is covered with an antibacterial coating.

5. System according to claim 1, wherein the bathing unit (16) is covered with a non-adherent coating.

6. System according to claim 2, wherein at least one membrane is provided being designed to decrease the amount of oxygen being present inside the volume (18).

7. System according to claim 6, wherein a membrane is located at the gas inlet (28) of the bathing unit (16).

8. System according to claim 6, wherein the membrane 44 forms part of the bathing unit 16.

9. System according to claim 6, wherein the membrane has a sorptive affinity for oxygen.

10. System according to claim 1, wherein a dispose unit (42) is arranged downstream the gas outlet (30).
